(19) United States
(12) Patent Application Publication
(10) Pub. No.: US 2025/0144403 A1
(43) Pub. Date: May 8, 2025

(54) BLOOD PUMP FOR SUPPORTING CARDIAC PERFORMANCE

(71) Applicants: MEDIZINISCHE UNIVERSITÄT WIEN, Wien (AT); CHARITÉ - UNIVERSITÄTSMEDIZIN BERLIN, Berlin (DE)

(72) Inventors: Marcus GRANEGGER, Perchtolsdorf (AT); Krishnaraj NARAYANASWAMY, Wien (AT); Andreas ESCHER, Wien (AT); Daniel ZIMPFER, Wien (AT); Ulrich KERTZSCHER, Berlin (DE)

(73) Assignees: MEDIZINISCHE UNIVERSITÄT WIEN, Wien (AT); CHARITÉ - UNIVERSITÄTSMEDIZIN BERLIN, Berlin (DE)

(21) Appl. No.: 18/838,134

(22) PCT Filed: Feb. 15, 2023
(86) PCT No.: PCT/IB2023/051358

§ 371 Date: Aug. 13, 2024

(30) Foreign Application Priority Data
Feb. 15, 2022 (EP) 22020051.3

(51) Int. Cl. A61M 60/81; A61M 60/122; A61M 60/226; A61M 60/419; A61M 60/824

(52) U.S. Cl. CPC A61M 60/81; A61M 60/122; A61M 60/226; A61M 60/419; A61M 60/824

(57) ABSTRACT

In a blood pump for assisting cardiac output, including a housing with a housing inlet and a housing outlet, wherein the housing forms a first pump housing and a second pump housing, a rotor which is mounted in the housing and is rotatable about an axis, wherein the rotor comprises a first pump stage with a first impeller arranged in the first pump housing and a second pump stage with a second impeller arranged in the second pump housing, wherein the first and the second pump stages are each formed as radial or diagonal pumps and through which the blood to be pumped from the housing inlet to the housing outlet can flow in succession, the first pump housing and the second pump housing are connected via a channel which runs radially outside a middle section of the housing arranged between the first and the second pump housing.

### CROSS-REFERENCE TO RELATED APPLICATIONS

The present application is a national phase application of PCT Application No. PCT/IB2023/051358, filed Feb. 15, 2023, entitled “BLOOD PUMP FOR SUPPORTING CARDIAC PERFORMANCE”, which claims the benefit of European Patent Application No. 22020051.3, filed Feb. 15, 2022, each of which is incorporated by reference in its entirety.

### BACKGROUND OF THE INVENTION

### 1. Field of the Invention

The invention refers to a blood pump for assisting cardiac output, including a housing with a housing inlet and a housing outlet, wherein the housing forms a first pump housing and a second pump housing, a rotor which is mounted in the housing and is rotatable about an axis, wherein the rotor comprises a first pump stage with a first impeller arranged in the first pump housing and a second pump stage with a second impeller arranged in the second pump housing, wherein the first and the second pump stages are each formed as radial or diagonal pumps and through which the blood to be pumped from the housing inlet to the housing outlet can flow in succession.

### 2. Description of the Related Art

Heart failure (HF) is the leading cause of death in Western countries. Modern mechanical circulatory support (MCS) devices promote survival and improve the quality of life of many HF patients. Rotodynamic blood pumps (RBPs) represent the vast majority of MCS devices implanted in the last decade, with survival rates above 70% after two years. Nevertheless, clinical outcomes remain limited by severe side effects, most of which are caused by suboptimal interactions between the pump system and blood. Only 20% of all RBP recipients remain free of life-threatening serious side effects, including bleeding or stroke, one year after implantation. Consequently, the quality of life of these patients remains severely limited and the cost-effectiveness of this therapy has not yet been achieved.

Modern implantable blood pumps are designed for adult patients suffering from HFrEF type heart failure, i.e. heart failure with reduced left ventricular ejection fraction. Such patients require implantable blood pumps that provide a comparatively high mean flow (4-5 l/min). The use of such pumps in low-flow applications (1-2.5 l/min) carries the risk of increased blood trauma and complication rate.

There is a need for a hemocompatible implantable blood pump that is suitable for patients in whom the pump is intended to provide comparatively low blood flow. Such cases include:
pediatric HF patients,
HFrEF patients with a lower degree of heart failure, especially NYHA stage 3-3b heart failure, to stop or reverse the progression of symptoms,
the large population of HF patients with preserved ejection fraction (HFpEF), a population for which no other effective long-term treatment option is currently available.

The article by Thamsen et al: “A two-stage rotary blood pump design with potentially lower blood trauma: a computational study” in Int J Artif Organs, 2016 Jun. 15; 39(4): 178-83, describes a two-stage blood pump for reducing blood damage. In this case, the pressure build-up is divided into two pump stages that can be flowed through one after the other, which leads to a reduction in the circumferential speeds of the impeller and thus reduced blood damage. Especially with small flows and comparatively high pressure differences (low specific speed), a two-stage design can lead to more efficient and blood-friendly operation. However, the embodiment proposed in Thamsen et al. does not allow miniaturization to the extent necessary for implantation. Another concept of a two-stage blood pump is described in document WO 2004/098677 A1. In the application with serial connection of the outlet of the first stage with the inlet of the second stage, the circumferential speeds can also be reduced. The concept is based on active regulation of the rotor position in the axial and/or radial direction by means of a “bearingless motor” and additional sensors for determining the rotor position. However, the integration of these sensors requires a larger installation space and thus leads to larger pump dimensions.

In summary, it should be noted that currently available blood pumps are either too large for implantation and/or have an increased risk of blood trauma and complication rates at lower blood flows in order to provide children or adults with less advanced heart defects. These observations can be explained by the fact that operating conditions with low pump flow rates at comparatively high pressures result in pump designs with low specific speeds—a key variable indicating that these operating conditions are incompatible with high hydraulic efficiencies. Consequently, comparatively high power is required to generate pressure and flow. This reduced efficiency is reflected in increased circumferential speeds, which in turn can lead to increased blood damage.

### SUMMARY OF THE INVENTION

The present invention therefore aims to provide a miniaturized implantable blood pump that has the potential to mitigate hemocompatibility-related complications. In particular, the invention is intended to address the need for a low flow blood pump for i) the pediatric population with heart failure and ii) adult patients with heart failure with either reduced (HFrEF) or preserved (HFpEF) ejection fraction.

To achieve this object, the invention essentially provides, in the case of a blood pump of the type mentioned at the outset, for the first pump housing and the second pump housing to be connected via a channel which runs radially outside a middle section of the housing arranged between the first and the second pump housing. Due to the fact that the blood to be transported from the first pump stage to the second pump stage is not conducted along the rotor, i.e. within the middle section of the housing, but outside the middle section, a short design of the blood pump in the axial direction is made possible. In particular, the middle section can be used as a rotor bearing due to the blood flow via the external channel. In this regard, a preferred embodiment of the invention provides for the rotor to be rotatably mounted in the middle section of the housing. Particularly preferably, the middle section of the housing forms a hydrodynamic bearing with the rotor. In this case, the middle section is formed with a preferably cylindrical inner surface, which interacts with a cylindrical rotor surface to form the hydrodynamic bearing. The bearing gap is, for example, 50-200 μm. To ensure almost wear-free function, the cylindrical surfaces can either be coated (Diamond Like Carbon, DLC) or made of suitable materials (polyetheretherketone (PEEK) or ceramics). In order to optimize the washing out of the hydrodynamic bearing, structures (e.g. spiral grooves) can be formed on one of the two cylindrical surfaces.

Due to the fact that the blood pump has two pump stages that can be flowed through in series, the pressure build-up is distributed over the two stages and allows operation at up to 30% lower speeds compared to single-stage pumps.

In a fluidically advantageous manner, it can be provided that the channel helically surrounds the middle section of the housing, wherein the direction of rotation of the helical shape is adapted to the direction of rotation of the rotor or the impellers. The helical channel may extend along an angle of, for example, 150-210° about the pump axis. The helical channel may provide radial space for driving and supporting the rotor.

According to a preferred embodiment of the invention, a fluidically favorable and space-saving arrangement of the channel can be achieved if the first pump housing has an annular region (outlet housing/volute) surrounding the first impeller, from which the channel tangentially leads away.

A flow-friendly discharge of the blood delivered by the pump is preferably achieved in that the second pump housing has an annular region surrounding the second impeller (outlet housing/volute), from which the housing outlet tangentially leads away.

Furthermore, the second pump housing can have an inlet housing section, which is spaced apart from the second impeller in the axial direction and into which the channel opens tangentially.

With regard to the electric drive of the blood pump, a preferred embodiment provides for the rotor to cooperate with a stator, which surrounds the middle section of the housing and has motor windings, in order to form an electric motor. In this case, the rotor may be provided with permanent magnets, the motor being designed as a brushless DC motor. The pump is thus driven by an electromagnetic motor integrated around the hydrodynamic bearing area. The motor windings of the stator are preferably formed by a single set of coils arranged one after the other in the circumferential direction, the winding axes of which are oriented radially toward the axis of rotation and are arranged in a plane running perpendicular to the axis of rotation. At the same time, the interaction of the stator with the rotor enables an axial suspension of the rotor on the basis of magnetic reluctance forces, which favors a miniaturized realization of the blood pump. Since the axial bearing of the rotor is effected solely by the reluctance forces acting between the stator with a single set of coils and the permanent magnets of the rotor, an additional axial bearing of the rotor can be dispensed with. The reluctance forces act mainly between the magnetic yoke of the stator and the permanent magnets of the rotor. The use of Halbach arrays as permanent magnets increases the axial reluctance forces. In addition, the reluctance force can be favored by a minimum distance between the permanent magnets of the rotor and the motor stator. Very thin wall thicknesses (<0.5 mm) in the middle section of the housing and the rotor are therefore advantageous for an optimal axial reluctance force.

Since no active control of the rotor position in radial and axial position is necessary, no sensors and no additional coils are required. This allows a compact design of the pump.

Due to the lack of active control of the axial rotor position, an axial force during operation leads to a corresponding axial displacement of the rotor position. In order to enable such a displacement, an axial gap is provided between the housing and the first and second impellers, respectively. However, the axial gaps increase the return flows in the pump and the axial size of the blood pump. In order to reduce this effect, a preferred embodiment of the invention provides that, in the rest position of the pump, the rotor is not arranged in an axial middle position between the corresponding housing surfaces, but is displaced in a direction towards the second pump stage. In structural terms, this preferred embodiment is characterized in that a first axial gap is provided between the first impeller and the first pump housing on the side facing away from the middle section of the housing, and a second axial gap is provided between the second impeller and the second pump housing on the side facing away from the middle section of the housing, and in that the rotor is held in its rest position in an axial position by the reluctance force acting between the motor windings and the permanent magnets of the rotor, so that the first gap is larger than the second gap.

Only during operation is the rotor displaced into the axial middle position due to the axial force acting on it, whereby the second gap is enlarged and the first gap is reduced.

In particular, the reluctance force is designed in such a way that the rotor assumes a middle position in which the first gap and the second gap are equal in size due to a mean pressure difference (between inlet and outlet of the entire pump) of 25-75 mmHg. The size of the first and second gaps is measured at the same radial distance from the axis of rotation.

Preferably, the housing inlet opens into the first pump housing on the side facing away from the middle section of the housing, and the inlet housing section opens into the second pump housing on the side facing away from the middle section of the housing, so that the axial forces acting on the rotor during operation act in opposite directions. In the second pump stage, however, there is a higher fluid pressure than in the first pump stage, resulting overall in a resulting axial force in the direction of the first pump stage.

Preferably, the blood pump according to the invention is designed to apply an average pressure difference of 35-150 mmHg.

In the middle position of the rotor, the size of the first and second gaps is 0.3-1 mm, in particular approx. 0.5 mm.

### BRIEF DESCRIPTION OF THE DRAWINGS

The invention is explained in more detail below with reference to an exemplary embodiment schematically illustrated in the drawing.

FIG. 1 shows an axial section of a blood pump according to the invention,

FIG. 2 shows an axial section along line II-II of FIG. 1, and

FIG. 3 shows a modified embodiment of the blood pump.

### DETAILED DESCRIPTION

The blood pump comprises a housing having an inlet tube 1, which slightly converges in the flow direction 3, and an outlet 2. The blood sucked in through the inlet tube 1 first passes through a first pump stage and then through a second pump stage. The first pump stage comprises a first pump housing 4, in which a first impeller 5 is arranged in a rotating manner. The impeller 5 drives the blood radially/diagonally outward into an annular region 6 surrounding the first impeller 5, from which a channel 7 tangentially leads away. Via the channel 7, the blood passes into an inlet housing section 8, from which it flows to the second pump stage, in which a second impeller 10 arranged in a second pump housing 9 rotates. The second impeller 10 drives the blood radially/diagonally outward into an annular region 11 of the second pump housing 9 surrounding the second impeller 10, from which the housing outlet 2 or the outlet tube 12 tangentially leads away.

The first and second pump stages are thus arranged one behind the other along the pump axis 13, the first impeller 5 and the second impeller 10 being arranged on the same rotor 14, which is rotatably mounted in a middle section 15 of the housing arranged between the first pump housing 4 and the second pump housing 9, forming a hydrodynamic bearing. Arranged around the middle section 15 of the housing are motor windings 16, which interact with the rotor 14 to form an electric motor. At the same time, this enables axial positioning of the rotor 14 on the basis of magnetic reluctance forces, wherein a first axial gap 17 is provided between the first impeller 5 and the housing and a second axial gap 18 is provided between the second impeller 10 and the housing.

It can also be seen that the channel 7 connecting the first pump housing 4 and the second pump housing 9 runs radially outside the middle section 15 of the housing.

The axially successive arrangement of the two pump stages in combination with a hydrodynamic bearing acting in the radial direction and a reluctance magnet suspension in the axial direction enables a miniaturized realization of the blood pump. The dimensions of the pump body (without inlet and outlet tube) can be approx. 2.5×2.5×2.5 cm or less.

Alternatively, a magnetic bearing or a mechanical bearing could be used instead of the hydrodynamic bearing.

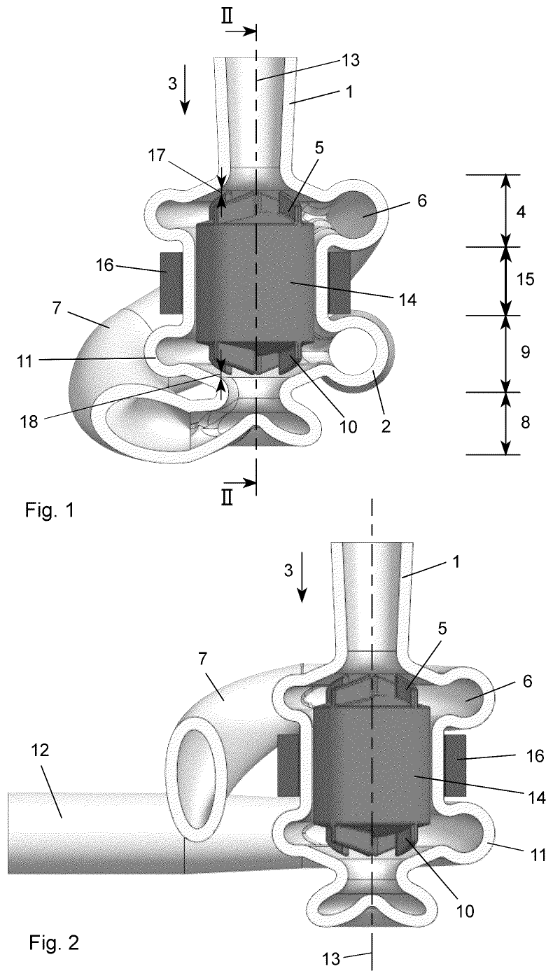

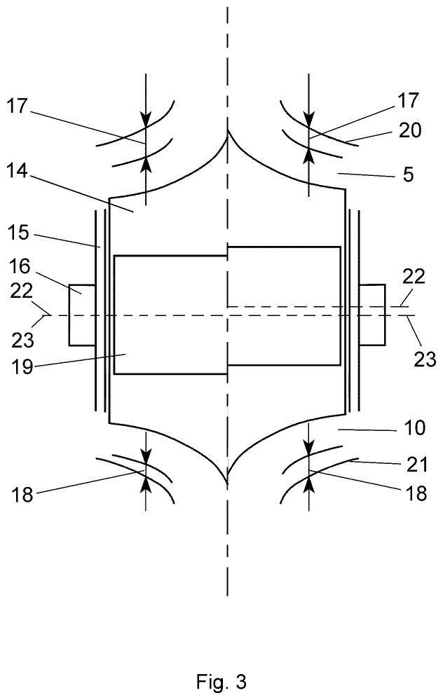

FIG. 3 shows a modified embodiment of the blood pump in a schematic partial representation, wherein the design corresponds to that of FIGS. 1 and 2. FIG. 3 shows only the axial mounting of the rotor 14 in interaction with the housing surfaces of the housing. In the embodiment shown, in the rest position, the rotor 14 is arranged eccentrically in the axial direction (left half of FIG. 3), so that the first gap 17 between the first impeller 5 and the housing surface 20 is larger than the second gap 18 between the second impeller 10 and the housing surface 21. The magnetic central line 22 of the permanent magnets 19 of the rotor 14 corresponds to the winding axis 23 of the motor windings 16.

During operation, due to the pressure difference between the first pump stage and the second pump stage, a resulting axial force acts on the rotor 14, which displaces the latter into the axial middle position and holds it there (right half of FIG. 4). In this middle position, the gaps 17 and 18 are substantially the same size and the magnetic central line 22 of the permanent magnets 19 is moved upward with respect to the winding axis 23 of the motor windings 16.

1-12. (canceled)

13. A blood pump for assisting cardiac output, comprising:
a housing with a housing inlet and a housing outlet, the housing forming a first pump housing and a second pump housing;
a rotor mounted in the housing and rotatable about an axis, the rotor including a first pump stage with a first impeller arranged in the first pump housing and a second pump stage with a second impeller arranged in the second pump housing;
wherein the first and the second pump stages are each formed as radial or diagonal pumps and through which the blood to be pumped from the housing inlet to the housing outlet can flow in succession; and
wherein the first pump housing and the second pump housing are connected via a channel which runs radially outside a middle section of the housing arranged between the first and the second pump housing.

14. The blood pump according to claim 13, wherein the channel helically surrounds the middle section of the housing.

15. The blood pump according to claim 13, wherein the first pump housing has an annular region surrounding the first impeller, from which the channel tangentially leads away.

16. The blood pump according to claim 13, wherein the second pump housing has an annular region surrounding the second impeller, from which the housing outlet tangentially leads away.

17. The blood pump according to claim 13, wherein the second pump housing has an inlet housing section, which is spaced apart from the second impeller in an axial direction and into which the channel opens tangentially.

18. The blood pump according to claim 13, wherein the rotor is rotatably mounted in the middle section of the housing.

19. The blood pump according to claim 18, wherein the middle section of the housing forms a hydrodynamic bearing with the rotor.

20. The blood pump according to claim 18, wherein the rotor is provided with permanent magnets and cooperates with a stator having motor windings and surrounding the middle section of the housing to form an electric motor.

21. The blood pump according to claim 20, wherein the motor windings of the stator are formed by a single set of coils arranged one after the other in the circumferential direction, the winding axes of which are oriented radially toward the axis of rotation and are arranged in a plane running perpendicular to the axis of rotation.

22. The blood pump according to claim 20, wherein the axial bearing of the rotor is realized solely by the reluctance force acting between the stator and the permanent magnets of the rotor.

23. The blood pump according to claim 20, wherein:
a first axial gap is provided between the first impeller and the first pump housing on the side facing away from the middle section of the housing;
a second axial gap is provided between the second impeller and the second pump housing on the side facing away from the middle section of the housing; and
the rotor is held in an axial position in a rest position of the rotor by the reluctance force acting between the motor windings and the permanent magnets of the rotor, so that the first gap is larger than the second gap.

24. The blood pump according to claim 23, wherein the reluctance force is configured so that the rotor assumes a middle position in which the first gap and the second gap are equal in size due to a pressure difference between the first and the second pump stages of 25-75 mmHg.
